(19) United States
(12) Patent Application Publication
(10) Pub. No.: US 2022/0047749 A1
(43) Pub. Date: Feb. 17, 2022

(54) FOOD PROCESSING AND/OR PACKAGING DEVICE AND METHOD FOR CLEANING SUCH DEVICE

(71) Applicant: Leybold GmbH, Köln (DE)

(72) Inventors: Peter Birch, Horsham West Sussex (GB); Clive Tunna, Bolney West Sussex (GB); John Mara, Evesham Worcestershire (GB); Hans-Jürgen Blum, Köln (DE)

(21) Appl. No.: 16/335,909

(22) PCT Filed: Oct. 10, 2017
(86) PCT No.: PCT/EP2017/075846

§ 371 Date: Mar. 22, 2019

(30) Foreign Application Priority Data
Nov. 3, 2016 (DE) 10 2016 221 560.4
Feb. 16, 2017 (EP) 17156508.8

(51) Int. Cl. A61L 2/26; B65B 55/02; B08B 7/00; B08B 9/027; A61L 2/10; A61L 2/04; A61L 2/20; A61L 2/22

(52) U.S. Cl. CPC A61L 2/26; B65B 55/027; B08B 7/0057; B08B 9/027; A61L 2/10; A61L 2/04; A61L 2202/17; A61L 2/22; B65B 2210/06; A61L 2202/121; A61L 2202/122; A61L 2202/11; A61L 2202/15; A61L 2/20

(57) ABSTRACT

A food processing and/or packaging device comprises at least one food chamber. Via a conduit system, the at least one food chamber is connected to a vacuum pump for generating vacuum in the at least one food chamber. For sterilizing the conduit system and/or the food chamber, a sterilization device is provided. The sterilization device comprises e.g. spray nozzles for introducing cleaning liquid into the conduit system. The cleaning liquid is separated by a liquid separator arranged upstream of the vacuum pump in flow direction.

### BACKGROUND

### 1. Field of the Disclosure

The disclosure relates to a food processing and/or packaging device and a method for cleaning such a device.

### 2. Discussion of the Background Art

The processing and particularly the packaging of food is often carried out under vacuum conditions. For this purpose, devices are known wherein a food chamber is connected, via a conduit system, to a vacuum pump. Within the food chamber, the food is e.g. processed and/or packaged. For instance, a food package will be evacuated with the aid of the vacuum pump. In such processes, which are at least temporarily performed under vacuum conditions, the problem exists that a liquid that is exposed to a reduced pressure will evaporate or turn into a fine aerosol. By means of the vacuum pump, the corresponding liquid particles will be sucked into the conduit system connected to the food chamber. At least a partial amount of these liquids will become deposited on surfaces of the conduit system. This will cause contamination of the conduit system. Further, the danger exists that an intrusion of bacteria via the air might also happen to cause a bacterial load. In the processing and/or packaging of food, however, this must be unconditionally avoided.

It is an object of the disclosure to provide a food processing and/or packaging device which can be cleaned in a simple manner. Further, it is an object of the disclosure to provide a method for cleaning such a device.

### SUMMARY

The food processing and/or packaging device according to the disclosure comprises at least one food chamber. Inside this chamber, the food items will be processed and/or packaged. Said at least one food chamber is connected via a conduit system, i.e. via tube conduits, to at least one vacuum pump so that an underpressure can be generated internally of said at least one food chamber, whereby the food is processed and/or packed under vacuum. According to the disclosure, at least one sterilization device is provided for sterilizing the conduit system which is possibly contaminated by components of the processed and/or packed food. Only the provision of a sterilization device makes it possible to guarantee a reliable eradication of bacteria also in the conduit system. Only by the sterilization—with the aid of the sterilization device—of the conduit system, i.e. of the inner sides of the tube conduits, it can be safeguarded that no bacteria or other contamination will intrude into the food package. Exclusively a reliable killing of bacteria and a complete sterilization of the conduit system will guarantee that corresponding contamination cannot penetrate from the conduit system into the vacuum chamber and then also into the food package. It is preferred that in addition to the sterilization of the conduit system, the food chamber may be sterilized, preferably by use of the same sterilization system.

Sterilization can be performed in various manners. According to a first preferred embodiment of the sterilization device, this device is designed in such a manner that the sterilizing is performed by means of a vacuum. For this purpose, an underpressure is generated in the conduit system. As a result, the bacteria will be killed. To safeguard a reliable elimination of the bacteria, it is preferred that an underpressure of at least 200 mbar is generated in the conduit system. This pressure is preferably maintained for a time period of at least 60 seconds. For generating a vacuum in the conduit system, the sterilization device comprises a closure device as well as a vacuum pump that is connected to the conduit system. With the aid of the closure device, which particularly comprises one or a plurality of valve devices, a tight sealing of the conduit system will be effected. By use of the vacuum pump, there will then be generated the required underpressure in the conduit system. The vacuum pump can be a separate vacuum pump or, according to a preferred embodiment of the disclosure, it can be the same vacuum pump by which an underpressure is generated in the food chamber.

Sterilization with the aid of underpressure can also be performed in the food chamber by evacuating the food chamber correspondingly.

According to a further preferred embodiment of the sterilization device, the sterilization of the conduit system and/or of the food chamber is performed with the aid of UV rays. For this purpose, the sterilization device comprises, according to a preferred embodiment, a UV radiation source for irradiating the conduit system and/or the food chamber. Preferably, use is made of UV radiation having a wavelength of about 255-280 nm. Irradiation is preferably performed with UV radiation intensity of at least 2000-8000 μW.s/cm².

A further possibility for sterilization of the conduit system or the food chamber resides in exposing the corresponding areas to a high voltage. For this purpose, the sterilization device comprises, according to a preferred embodiment, a high-voltage source. It is preferred that the voltage is at least 300 V and that the corresponding areas are subjected to this voltage for a time period of at least 60 seconds.

A further possibility for sterilization consists in heating the corresponding areas and respectively performing a hot treatment on them, again in order to kill the bacteria. For this purpose, the sterilization device comprises, according to a preferred embodiment, a heating device. With the aid of the heating device, the corresponding areas such as particularly the conduit system and/or the food chamber are subjected to a temperature of at least 100° C. The heating or hot treatment of the corresponding areas is preferably performed over a time period of at least 60 seconds.

Optionally, the above described different sterilization devices can be partially or completely combined with each other. Particularly, different sterilization devices can be arranged in certain areas in dependence on the accessibility of the areas. For instance, in areas that are accessible only with difficulty, heating devices in the form of heating coils, heating wires or the like can also be integrated into the corresponding device, such as e.g. into a branch conduit of a conduit system.

According to a particularly preferred embodiment of the disclosure, the UV radiation source and/or the high-voltage source and/or the heating device at least partially surround a tube conduit of the conduit system. With particular preference, the corresponding device is of a ring-shaped design and thus fully surrounds the tube conduit. According to a further preferred embodiment, the UV radiation source and/or the high-voltage source and/or the heating device can arranged to be moved or shifted in longitudinal direction of the tube conduit. Particularly, this makes it possible to sterilize a tube by shifting the corresponding ring-shaped device in longitudinal direction.

According to a further embodiment, the radiation source and/or the high-voltage source and/or the heating device are designed to be arranged internally of the conduit system and are movable or shiftable within it. Preferably, these devices are realized in the form of a movable robot designed to move through the conduit system.

According to a further preferred embodiment, the sterilization device comprises a gas supply device. With the aid of the gas supply device, a gas suited for sterilization of the conduit systems, such as e.g. ozone, can be supplied to the conduit system. Other suitable gases are fluorine, chlorine, bromine, iodine etc. Of course, use can be made also of mixtures of these gases, or other gases with antibacterial properties. The process of pumping the gases through the conduit system and/or the food chamber can be performed by a separate pump or preferably with the aid of the existing pump. Optionally, the vacuum pump has a separator arranged upstream of it in flow direction for preventing the intrusion of impurities into the vacuum pump.

Further, the gas supply device can be combined with one or a plurality of other sterilization devices.

For cleaning the conduit system and/or the food chamber, there is provided, according to a further preferred embodiment, a liquid supply device for supply of cleaning liquid. The liquid supply device is connected to at least one spraying nozzle. The spraying nozzle serves for supplying the cleaning liquid into the conduit system. According to the disclosure, there is further provided, in addition to said at least one spraying nozzle, a liquid separator arranged upstream of the vacuum pump in flow direction. The liquid separator serves for separating the cleaning liquid so as to avoid intrusion of the cleaning liquid into the vacuum pump. In this arrangement, it is preferred to use liquid separators that will separate more than 98%, preferably more than 99% of the liquid so that, at the most, only minimal amounts of liquid can leak into the vacuum pump. It is also acceptable to allow the cleaning liquid to pass through the vacuum pump if it is able to handle the liquid flow, in this case a liquid separator would not be required in front of the pump but might be used in the exhaust of the vacuum pump.

According to a preferred embodiment of the disclosure, the food processing and/or packaging device is operated—according to the cleaning method of the disclosure—in such a manner that, first, the food processing and/or packaging process will be interrupted or terminated. Subsequently, the vacuum pump will be switched off or at least be reduced, preferably considerably so, with respect to its conveying quantity. Then, the cleaning liquid will be supplied into the conduit system, particularly via said at least one spraying nozzle. In a next step, according to a particularly preferred embodiment, in order to guarantee a distribution of the generated spraying nozzle in the entire conduit system, the vacuum pump will again be switched on with reduced conveying volume and respectively be caused to continue its operation with reduced conveying volume. Then, the supply of cleaning liquid into the conduit system will be interrupted. Subsequently, the pump will be fully opened again and respectively be operated again with higher conveying quantity so that the spray mist or aerosol of cleaning liquid will be pumped off from the conduit system. In the process, the liquid will be separated in the liquid separator that is arranged, particularly directly, upstream of the vacuum pump as viewed in flow direction.

According to a particularly preferred embodiment, the spraying nozzles are designed to generate a spray mist or an aerosol of cleaning liquid. Herein, the particle size of the liquid is preferably smaller than 100 μm, particularly smaller than 50 μm.

Further, it is preferred that said at least one spraying nozzle is arranged in the area of an outlet of the food chamber. In case that a plurality of food chambers are provided, it is particularly preferred that, for each food chamber, respectively one spraying nozzle is provided at the corresponding outlet where the food chamber is connected to the conduit system. Thereby, it is safeguarded that the cleaning liquid will be distributed in the entire conduit system.

According to a preferred embodiment of the disclosure, the liquid supply device comprises a liquid pump. This pump is preferably adapted to generate a pressure in the range from 50 to 100 bar, and/or the pump preferably has a conveying capacity in the range from 1 to 10 l/h. Further, it is preferred that the liquid supply device comprises a reservoir for the cleaning liquid. Preferably, the reservoir is connected to the liquid pump. By use of a corresponding sensor and, optionally, a control unit connected thereto, it can be safeguarded that this liquid pump will be operated only if a sufficient quantity of cleaning liquid exists in the reservoir.

For performing the method of the disclosure for sterilization with the aid of a liquid introduced via spraying nozzles, it is preferred that, for distributing the spray mist of cleaning liquid, the vacuum pump is operated with reduced conveying quantity. This could be performed by throttling the vacuum pump with the aid of a control unit. According to a particularly preferred embodiment, the food processing and/or packaging device comprises a valve. This valve is preferably arranged upstream of the vacuum pump in flow direction or is integrated in the vacuum pump e.g. at the inlet of the latter. In correspondence to the position of the valve, the conveying quantity of the vacuum pump can be changed without changing e.g. the rotational speed of the vacuum pump. As a valve, preferred use is made of a butterfly valve. According to a particularly preferred embodiment, the butterfly valve is of the type with positive opening and respectively with a hole. This has the result that, even in the fully closed state of the throttle flap, a passage opening will remain. Particularly, the use of such a valve has the advantage that the valve can be switched in a simple manner. In the closed state of the valve, a residual opening will remain so that a small conveying quantity will continue to be conveyed by the vacuum pump. When cleaning liquid is supplied to the conduit system, the valve is switched into this position. In the fully opened state of the throttle flap, the vacuum pump will convey the maximum quantity. In general terms, changing of the conveying quantity of the vacuum pump is performed by controlling the valve in a corresponding manner, wherein the above described use of a butterfly valve with forced opening and the simplified control are preferred. The reduced conveying quantity can also be achieved by reducing the rotational speed of the vacuum pump where such an action will have the result of reduced conveying quantity.

According to a preferred embodiment of the food processing and/or packaging device, there is further provided at least one particle and/or liquid filter. Preferably, said at least one particle and/or liquid filter is arranged in the area of the outlet of the food chamber. Particularly, the corresponding particle and/or liquid filter is arranged immediately behind the outlet of the food chamber. According to a preferred embodiment, the spraying nozzle is arranged between the outlet of the food chamber and the particle and/or liquid filter. If the device comprises a plurality of food chambers, it is again preferred that, for each food chamber, one particle and/or liquid filter is arranged at the outlet and is situated, in flow direction, behind the spraying nozzle that is preferably provided for each food chamber.

When using the preferred device comprising at least one particle and/or liquid filter, it is preferred that this filter be cleaned prior to performing the cleaning method.

The disclosure further relates to a method for cleaning a food processing and/or packaging device of the design described above. In a first step of this method, the vacuum pump will be switched off or throttled, i.e. its performance and respectively its conveying volume will be reduced. This is followed by interruption and respectively termination of the food processing and/or packaging process in the food chamber. Then, according to the disclosure, a sterilization device is activated for a sterilization period. This time period depends particularly on the sterilization device and respectively the combination of sterilization devices that is/are used. Further, the time period depends e.g. on the strength of the UV radiation and the level of the voltage, the temperature etc.

In the sterilization process by generating a vacuum, activation is performed by closing a closure device and by subsequent generation of vacuum in the conduit system and/or the food chamber. With respect to the provision of a UV radiation source and/or a high-voltage source and/or a heating device, activation is performed by switching on the corresponding device and optionally by moving the corresponding device in longitudinal and/or circumferential direction of the tube conduits of the conduit system.

With respect to the supply of a gas for sterilization or a cleaning liquid for sterilization, activation is performed by a corresponding supply of the medium.

The described process would also apply to other markets where there is the possibility of microbiological contamination of the vacuum pipework.

The disclosure will be described in greater detail hereunder with reference to the accompanying drawing.

### BRIEF DESCRIPTION OF THE DRAWINGS

In the various Figures of the drawing, the following is shown:

FIG. 1 is a schematic view of a first embodiment of a configuration of a food processing and/or packaging device;

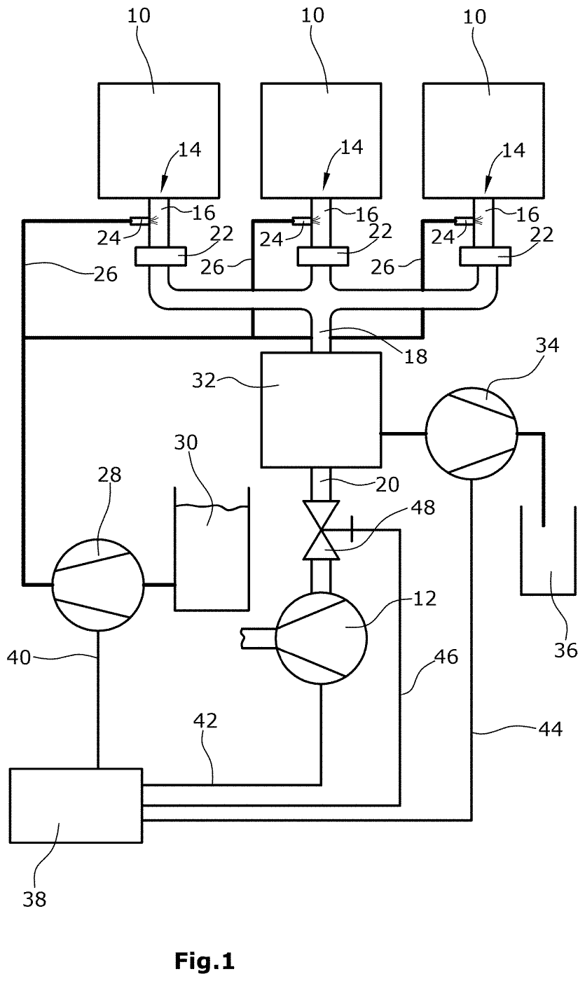

FIG. 2 is a schematic view of a second embodiment of a configuration of a food processing and/or packaging device; and

FIG. 3 is a schematic view of a third embodiment of a configuration of a food processing and/or packaging device.

### DETAILED DESCRIPTION OF THE PREFERRED EMBODIMENT

In the illustrated exemplary embodiment of the food processing and/or packaging device, three food chambers 10 are shown. In the interior of the food chambers 10, a vacuum can be generated, by use of a pump 12, for the processing and/or packaging of food. For this purpose, the respective outlets 14 of the food chambers 10 are connected to the vacuum pump 12 via tube conduits 16,18,20. The tube conduits 16 of the conduit system have respectively one particle and liquid filter 22 arranged in them, wherein these filters 22 serve primarily for the filtering of particles so as to avoid intrusion of particles into the vacuum pump 12.

In the illustrated exemplary embodiment, for each tube conduit 16 of the conduit system, a spraying nozzle 24 is provided in the area of outlet 14. Via the spraying nozzles 24, a cleaning liquid can be supplied to the conduit system 16,18,20 for sterilizing the conduit system. For this purpose, the spraying nozzles 24 are connected via a common conduit 26 to a liquid pump 28. Liquid pump 28 is connected to a reservoir 30 containing a supply of cleaning liquid.

Further, a liquid separator 32 is provided in the tube conduit 18 of the conduit system, said liquid separator 32 being arranged upstream of the vacuum pump 12 as seen in flow direction. Said liquid separator serves for separating the cleaning liquid. For this purpose, liquid separator 32 is connected to a pump 34 operative to pump the spent cleaning liquid into a collection container 36.

For performing the cleaning method of the disclosure, particularly automatically, there is further provided a control device 38. This control device is connected to pump 28 via a control line 40, to vacuum pump 12 via a control line 42, to liquid pump 34 a control line 44, and to a valve 48 via a control line 46. Said valve 48, which might be a modified butterfly valve, is arranged in tube conduit 20 upstream of the vacuum pump 12 as seen in flow direction.

According to a preferred manner of practicing the cleaning method of the disclosure, it is provided that, first, the packaging or the like process performed on the food in the food chambers will be interrupted or terminated. Subsequently, the vacuum pump 12 will be throttled by controlling the valve 48, slowing the rotational speed, or the vacuum pump 12 will be switched off entirely. This is followed by cleaning the filters 22, e.g. by dismounting, exchanging or cleaning them. For dismounting or cleaning the filters 22, it is preferred that the vacuum pump 12 will be switched off entirely. Optionally, however, the cleaning method of the disclosure can be carried out also without a previous cleaning of the filters 22 because, depending on the circumstances, these do not need to be cleaned prior to each cleaning cycle. In case that the vacuum pump 12 had been switched off for cleaning of the filters 22, the vacuum pump 12 will now be switched on in the closed state of the throttle valve 48, wherein the valve 48 is switched off in such a manner that there will always remain a forced opening or residual opening, resulting in a continued conveyance of a small conveying quantity by the vacuum pump 12.

In the next step, the liquid pump 28 will be switched on, particularly by control device 38, so that cleaning liquid will be sprayed into the conduit system 16,18,20 via the nozzles 24. Due to the still continuing—although small—conveying quantity conveyed by vacuum pump 12, a uniform and particularly complete distribution of the spray mist of the cleaning liquid will take place in the entire conduit system 16,18,20. After the cleaning period of the conduit system 16,18,20 has been achieved (e.g., 8 hours), the pump 28 will be switched off again. Then, the valve 48 will be fully opened, with a resultant increase of the conveying quantity of vacuum pump 12. Thereby, the spray mist of cleaning liquid will be removed again from the entire conduit system 16,18,20. In the process, the cleaning liquid itself will be filtered out by the filter 32. The filtered-out cleaning liquid will be pumped into collection container 36 with the aid of pump 34. In this regard, the pump 34, as far as it also has certain dry-running properties, will be switched on already at the start of the cleaning step, or only toward the end of the cleaning step, e.g. simultaneously with the opening of valve 48. Further, the pump 34 can be connected to a corresponding humidity sensor in the filter so that the pump 34 will change into the switch-on state in a self-acting manner.

Instead of supplying a cleaning liquid via nozzles 24, it is also possible to supply a cleaning gas to the tube conduits 16. The latter supply can be performed via corresponding supply nozzles or inlets which, depending on the given circumstances, are preferably arranged in the area of the nozzles 24. In a sterilization device of this configuration, the liquid separator 32 can be optionally omitted or be replaced by a simple filter. Accordingly, in this case, the pump 34 and the collection container 36 are not required anymore. Further, for supply of gas, it is possible to provide —instead of a supply pump 28 connected to a gas supply container 30—a pressurized gas container so that, by opening corresponding valves, gas will be directly supplied to the conduits 16. In this case, it would not be a pump 28 but a corresponding valve of a gas container that would be controlled by control device 38.

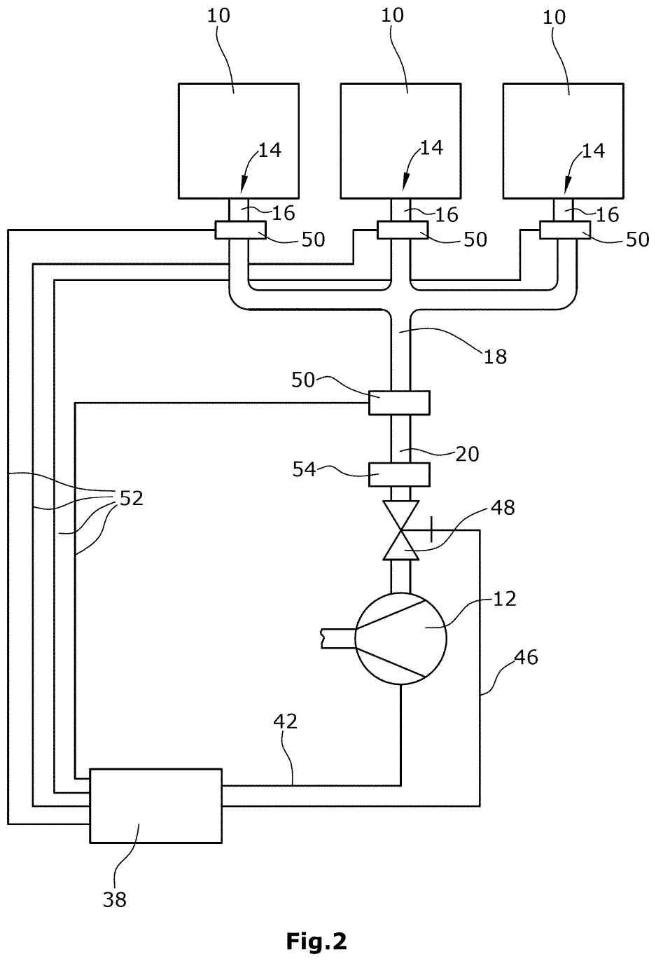

In the further preferred embodiment shown in FIG. 2, similar and identical components are designated by the same reference numerals as above.

The embodiment shown in FIG. 2 relates to the sterilizing of conduits 16 of the conduit system with the aid of UV radiation sources and/or high-voltage sources and/or heating devices. These are schematically represented by component 50. Component 50 particularly is a ring-shaped component surrounding the respective tube conduit 16. Herein, component 50 can be a UV radiation source and/or a high-voltage source and/or a heating device or a combination of individual ones of these devices. These are preferably arranged to be displaced in longitudinal direction of the tube conduits. Control of the devices 50 is performed with the aid of a control unit 38 which is electrically connected to the devices 50 via control lines 52.

In the illustrated exemplary embodiment, a filtering device 54 is arranged in flow direction upstream of the vacuum pump 12 for avoiding an intrusion of particles into vacuum pump 12.

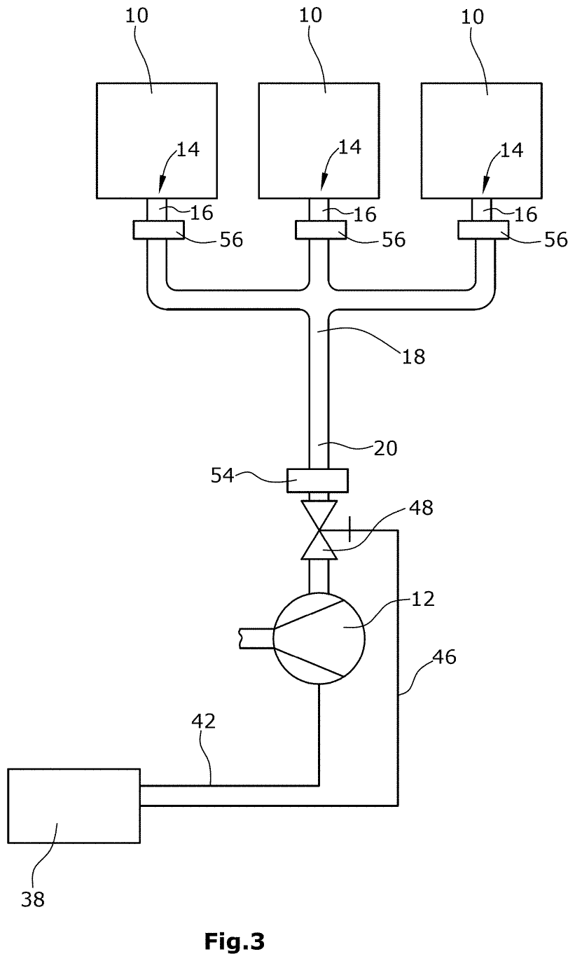

In a still further preferred embodiment (FIG. 3), similar and identical components are again designated by the same reference numerals. In this embodiment, the sterilization of the conduit system 16,18,20 is carried out by vacuum. For this purpose, valve devices 56 are provided in the area of the outlets of food chamber 10. For sterilization, the valves will be closed so that, with the aid of pump 12, a vacuum can be generated in the tube conduit system 16,18,20. In this embodiment, filter 54 can be optionally omitted.

1. A food processing and/or packaging device, comprising
at least one food chamber in which food is processed and/or packed,
a conduit system connected to the at least one food chamber,
a vacuum pump connected to the conduit system, for generating vacuum in the at least one food chamber while the food is processed and/or packed, and
at least one sterilization device for sterilizing the conduit system being possibly contaminated by components of the processed and/or packed food.

2. The food processing and/or packaging device according to claim 1, wherein the sterilization device comprises a closure device for closing the conduit system and/or the food chamber, and a vacuum pump for generating vacuum in the conduit system and/or the food chamber.

3. The food processing and/or packaging device according to claim 1, wherein the sterilization device comprises a UV radiation source for irradiation of the conduit system and/or the food chamber.

4. The food processing and/or packaging device according to claim 1, wherein the sterilization device comprises a high-voltage source for sterilizing the conduit system and/or the food chamber.

5. The food processing and/or packaging device according to claim 1, wherein the sterilization device comprises a heating device for heating the conduit system and/or the food chamber.

6. The food processing and/or packaging device according to claim 3, wherein the UV radiation source and/or the high-voltage source and/or the heating device at least partially surround a tube conduit of the conduit system, with the UV radiation source and/or the high-voltage source and/or the heating device surrounding a tube conduit of the conduit system preferably in a ring-shaped configuration.

7. The food processing and/or packaging device according to claim 3, wherein the UV radiation source and/or the high-voltage source and/or the heating device are displaceable in longitudinal direction of the tube conduit.

8. The food processing and/or packaging device according to claim 1, wherein the sterilization device comprises a gas supply device for supply of gases, particularly antibacterial gases.

9. The food processing and/or packaging device according to claim 1, further comprising by at least one spraying nozzle connected to a liquid supply system, said spraying nozzle being connected to the conduit system for supply of cleaning liquid, and by a liquid separator arranged upstream of the vacuum pump in flow direction, for separating the cleaning liquid introduced into the conduit system via the at least one spraying nozzle.

10. The food processing and/or packaging device according to claim 9, wherein a valve is connected upstream of the vacuum pump for varying the conveying volume of the vacuum pump.

11. A method for cleaning a food processing and/or packaging device according to claim 1, comprising the steps of:
switching off or throttling the vacuum pump,
interrupting and respectively terminating the food processing and/or food packaging process in the food chamber, and
activation of the sterilization device for a sterilization period.

12. The method for cleaning a food processing and/or packaging device according to claim 11, wherein said activation is performed by closing a valve device and generating a vacuum in the conduit system and/or in the food chamber.

13. The method for cleaning a food processing and/or packaging device according to claim 11, wherein said activation is performed by switching on a the UV radiation source and/or the high-voltage source and/or the heating device.

14. The method for cleaning a food processing and/or packaging device according to claim 13, wherein the UV radiation source and/or the high-voltage source and/or the heating device are moved particularly in longitudinal and circumferential direction of the tube conduit of the conduit system.

15. The method for cleaning a food processing and/or packaging device according to claim 11, wherein said activation of the sterilization device is performed by the following steps:
supplying cleaning liquid into the conduit system,
switching off the vacuum pump or continuing the operation of the vacuum pump with reduced conveying volume,
interrupting the supply of cleaning liquid into the conduit system, and
operating the vacuum pump with increased conveying volume.
